(19) United States
(12) Patent Application Publication
(10) Pub. No.: US 2003/0185889 A1
(43) Pub. Date: Oct. 2, 2003

(54) Colloidal nanosilver solution and method for making the same

(76) Inventors: Jixiong Yan, Wuhan (CN); Jiachong Cheng, Beijing (CN)

Correspondence Address: VENABLE, BAETJER, HOWARD AND CIVILETTI, LLP, P.O. BOX 34385, WASHINGTON, DC, 20043-9998, US

(21) Appl. No.: 10/106,053

(22) Filed: Mar. 27, 2002

(51) Int. Cl. A61K 9/14

(52) U.S. Cl. 424/484

(57) ABSTRACT

The present invention provides a colloidal nanosilver solution which contains nanosilver particles having diameters between 1 nm and 100 nm. The silver content in the colloidal solution is between 0.001% to 0.4% by weight. The colloidal nanosilver solution also contains a gelling agent which includes, but is not limited to, starch or its derivative, cellulose or its derivative, polymer or copolymer of acrylate or acrylate derivative, polyvinyl pyrrolidone, alginic acid, and xanthogenated gel. The present invention also provides a method for making the colloidal nanosilver solution. The colloidal nanosilver solution prepared by this method does not contain any toxic or impure substances.

### FIELD OF THE INVENTION

The present invention relates to a colloidal nanosilver solution containing nanosilver particles with sizes ranged from 1 to 100 nm in diameter. The silver content in the colloidal nanosilver solution is about 0.001% to 0.4% by weight. The colloidal nanosilver solution also contains a gelling agent which includes, but is not limited to, starch or its derivatives, cellulose or its derivative, polymer or copolymer of acrylate or acrylate derivative, polyvinyl pyrrolidone, alginic acid, and xanthogenated gel. The colloidal nanosilver solution is characterized by not containing toxic or impure substances and is suitable for use in sanitation, disinfection, or as antimicrobial agent for treatment of patients. The present invention also relates to a method for making the colloidal nanosilver solution by interacting silver oxide first with ammonia water then with hydrazine hydrate.

### DESCRIPTION OF THE RELATED ART

It has been known for centuries that silver possesses germicidal properties and has been employed as germicide before modern antibiotics were developed. During those days, users would shave silver particles into their drinking water, or submerge whole silver pieces in the drinking water, for the purpose of ingesting silver by drinking the water. Even after the onset of modem antibiotics era, silver remains to be used as antimicrobial agent, particularly because microorganisms treated by silver do not acquire resistance to the metals, while the conventional antibiotics often induce the formation of antibiotic-resistant microorganisms.

Silver is a safe and effective antimicrobial metal. In the late eighteenth century, western scientists confirmed that silver, which had been used in oriental medicine for centuries, was an effective antibacterial agent. Scientists also knew that the human body fluid is colloidal. Therefore, colloidal silver had been used for antibacterial purposes in the human body. By the early nineteenth century, colloidal silver was considered the best antibacterial agent until the discovery of the antibiotics. Due to the potency and revenue-driven advantages of antibiotics, the antibiotics gradually substituted colloidal silver as the main choice for antibacterial agent. However, thirty years into the discovery of the antibiotics, scientists began to discover that antibiotics induced the development of antibiotic-resistant bacterial strains which significantly affected the efficiency of antibiotics. Therefore, since 1870s, silver has again been recognized as a preferred antibacterial use, particularly due to its non-toxic and non-induction of bacterial-resistant characteristics.

There are many reasons why administering silver suspended in solution (e.g., colloidal silver) would enhance an individual's health. It is possible that such a solution operates to inhibit the growth of bacteria, fungi, viruses, and other unwanted organisms, as well as eradicating such existing bacteria, fungi, viruses, and other organisms. It is also possible that a solution of silver can have an anti-inflammatory effect, sufficient to reduce symptoms of asthma. Silver in solution might also act in a similar fashion to a homeopathic remedy.

There have been numerous attempts to produce silver-based solutions, including colloidal silver. However, many of the silver-based products fail to maintain the silver particles in suspension, either because the silver solution is not a true colloid or because it is otherwise unstable. When the suspension of the silver particles fails, the particles fall to the bottom of the solution, thereby reducing the solution's concentration of silver and rendering it less effective.

In the invention to be presented in the following sections, a colloidal nanosilver solution will be disclosed. The colloidal nanosilver solution of the present invention can maintain the colloidal nanosilver particles in suspension for a long period of time. It also has the advantages of not containing toxic or impure substances so that it is particularly suitable for medicinal and healthcare use.

### SUMMARY OF THE INVENTION

The present invention provides a colloidal nanosilver solution containing nanosilver particles with sizes ranged between 1 nm and 100 nm in diameter, the silver content in the colloidal nanosilver solution is about 0.00% to 0.4% by weight.

The colloidal nanosilver solution also comprises a gelling agent, which is starch or its derivative, cellulose or its derivative, polymer or copolymer of acrylate or acrylate derivative, polyvinyl pyrrolidone, alginic acid, or xanthogenated gel. Examples of starch derivative include, but are not limited to, sodium carboxymethyl starch, hydroxyethyl starch, and pregelatinized starch. Examples of cellulose derivative include, but are not limited to, methyl cellulose, hydroxypropyl cellulose, hydroxypropyl methyl cellulose, and hydroxyethyl cellulose. An example of polymer or copolymer of acrylate derivative is carbomer, which is a carboxy vinyl polymer. Carbomer generally are high molecular weight (“MW”) polymers (MW above 1,000,000). Carbomer is commercially available. B. F. Goodrich Company currently sells carbomer using the tradename of Carbopol. Carbopol 934P has a MW of about 3,000,000 and Carbopol 940 is about 4,000,000. The preferred Carbopol is Carbopol 934P. The preferred concentration of the gelling agent is at about 0.2 to 5% by weight of the total solution.

The colloidal nanosilver solution has antimicrobial activity, particularly for inhibiting growth of bacteria, fungi, or chlamydia. Examples of microorganisms which can be inhibited by the colloidal nanosilver solution include, but are not limited to, Escherichia coli, Methicillin resistant Staphylococcus aureus, Chlamydia trachomatis, Providencia stuartii, Vibrio vulnificus, Pneumobacillus, Nitrate-negative bacillus, Staphylococcus aureus, Candida albicans, Bacillus cloacae, Bacillus allantoides, Morgan's bacillus (Salmonella morgani), Pseudomonas maltophila, Pseudomonas aeruginosa, Neisseria gonorrhoeae, Bacillus subtilis, Bacillus foecalis alkaligenes, Streptococcus hemolyticus B, Citrobacter, and Salmonella paratyphi C.

The present invention also provides a method for making the colloidal nanosilver solution. The method includes the following steps: (1) dissolving silver oxide (Ag₂O) in ammonia water (NH₃.H₂O) to form a solution containing silver ammino ion [Ag(NH³)⁺]; (2) dissolving a gelling agent in water to form a gelling medium; (3) mixing the silver ammino ion-containing solution with the gelling medium to form a colloidal nanosilver ammino ion-containing solution; and (4) mixing the colloidal nanosilver ammino ion-containing solution with hydrazine hydrate (NH₂NH₂.H₂O) to form the colloidal nanosilver solution. The ammonia water used in step (1) is preferred to be at a concentration of 28% by weight. Also, the silver oxide and the ammonium water in step (1) is preferred to be at a ratio of about 1:7 to about 1:10 (w/v). In addition, the silver oxide and the hydrazine hydrate in step (4) is preferred to be at a ratio of about 1:0.087 to about 1:0.26 (w/v).

It is preferred that the colloidal nanosilver ammino ion-containing solution is mixed with hydrazine hydrate (NH₂NH₂.H₂O) at about 0 to 45° C. for about 0.5 to 2 hours. Also, after the formation of the colloidal nanosilver solution, it is preferred to let the colloidal nanosilver solution be in contact with air for about 0.5 to 5 hours.

The colloidal nanosilver solution can be used as an antibacterial or antifingal agent for treatment of patients with bum and scald-related skin infection, wound-related skin infection, dermal or mucosal bacterial or fungal infection, surgery cut infection, vaginitis, and acne-related infection, by applying or spraying the solution onto the wounds. It can also be used as a disinfectant or sanitary agent to clean areas in need of disinfection or sanitation.

### DETAILED DESCRIPTION OF THE INVENTION

The present invention provides a colloidal nanosilver solution which contains nanosilver particles having diameters with sizes ranged between 1 nm and 100 nm. A colloid is a gelatinous or mucinous dispersion medium that consists of particles which are larger than an ordinary crystalloid molecule, but are not large enough to settle out under the influence of gravity. These particles normally range in size from 1 to 100 nm. There are generally two kinds of colloids: (1) Suspension colloids (suspensoids), in which the dispersion medium consists of particles that are insoluble, such as a metal, and the dispersion medium may be gaseous, liquid, or solid. (2) Emulsion colloids (emulsoids), in which the dispersion medium is usually water and the disperse phase consists of highly complex organic substances, such as starch or glue, which absorb much water, swell, and become uniformly distributed throughout the dispersion medium. The suspension colloids tend to be less stable than the emulsion colloids. The colloidal nanosilver solution of the present invention is a hybrid of both the suspension and the emulsion colloids.

The colloidal nanosilver solution of the present invention is further characterized by its non-toxic and purity nature, as well as its stability. The silver content in the colloidal solution is between 0.001% to 0.4% by weight. It is also stable at room temperature (about 25° C. or 77° F.) for at least 110 days. Because of these characteristics, the colloidal nanosilver solution is particularly suitable for use in healthcare related matters such as sanitization and disinfection.

The colloidal nanosilver solution of the present invention can be used in sanitary products, which include, but are not limited to, solutions for cleansing agents for clothing, women hygiene, acne or pimples, and soaking solution for tooth brush. It can also be used in healthcare products, which include, but are not limited to, treating patients with all kinds of injuries and/or burns, bacterial and fungal infections (including gynecological infections such as vaginitis), gastrointestinal bacterial infection, and sexually transmitted diseases. In addition, the colloidal nanosilver solution of the present invention can be used in industrial products, which include, but are not limited to, food preservatives especially for fruits and vegetables, drinking water disinfectants, paper and construction filling materials preservation (especially to prevent mold formation).

The colloidal nanosilver solution of the present invention possesses a broad spectrum of antibacterial and antifungal ability. It can kill and suppress growth of bacteria and fungi, such as Escherichia coli, Methicillin resistant Staphylococcus aureus, Chlamydia trachomatis, Providencia stuartii, Vibrio vulnificus, Pneumobacillus, Nitrate-negative bacillus, Staphylococcus aureus, Candida albicans, Bacillus cloacae, Bacillus allantoides, Morgan's bacillus (Salmonella morgani), Pseudomonas maltophila, Pseudomonas aeruginosa, Neisseria gonorrhoeae, Bacillus subtilis, Bacillus foecalis alkaligenes, Streptococcus hemolyticus B, Citrobacter, and Salmonella paratyphi C.

The antibacterial and antifungal activity of the colloidal nanosilver solution of the present invention has advantage over the conventional antibiotics in killing and suppressing bacterial growth, as it does not induce drug-related resistance in the bacterial or fungal strains.

Conventionally, a colloidal silver solution is prepared by reducing silver nitrate (AgNO₃) to metallic silver with reducing agent such as glucose, ascorbic acid, hydrazine hydrate, hydrazine sulfate, and formaldehyde. The term “colloidal silver solution” as used in this context refers to a colloidal solution containing silver particles where the sizes of the silver particles are not necessarily within the nanometer range in the “colloidal nanosilver solution” as described in the present invention.

Under this preparation method, other than silver, oxidized products of the reducing agents, which are possibly toxic, are generated. The presence of these oxidized products not only affect the purity of the colloidal silver solution but also make the colloidal silver solution unsuitable for use in healthcare related industry due to its toxicity. Also, although the oxidized products of the reducing agents can be removed from the colloidal silver solution by conventional methods, such as dialysis, the method of dialysis involves excessive steps which not only is time-consuming but also adds more difficulties and expenses to the industrial-scale manufacturing process.

To avoid producing unwanted toxic products, at least two methods are disclosed which produce a colloidal silver solution containing harmless side products from the reducing agents. For example:

(1) Reacting silver oxide (Ag₂O) with hydrogen gas to form metallic silver and water:

Ag₂O+H₂→2Ag+H₂O

(2) Reacting silver oxide (Ag₂O) with hydrazine hydrate (NH₂NH₂.H₂O) to form metallic silver, nitrogen gas, and water.

2Ag₂O+NH₂NH₂.H₂O→4Ag+N₂+3H₂O

Because the above reactions produce metallic silver, nitrogen gas, and water, which are non-toxic in nature so that no additional steps are necessary for removing the unwanted toxic products, theoretically, they should be suitable for the production of colloidal silver solution. However, the reactions as shown above are not practical in manufacturing industrial-scale colloidal nanosilver solution. For example, in the reaction as described in (1), which requires the silver oxide to interact with hydrogen gas, a multiphase reaction is involved which make it very difficult to carry out. See V. Kohlschuetter Strassburg (Z. Elektrochem., 14, 49-63. CA: 2: 1379-1380). When the silver oxide and hydrogen are sealed in a glass tube and reacted at 18° C. or lower, the reduction reaction takes place very slowly. On the other hand, if the reaction is carried out at 60° C. or lower, the hydrogen gas is discharged into the saturated silver oxide solution, which results in yielding a colloidal silver solution with silver particles partially in suspension and partially precipitated. A colloidal silver solution prepared in this way is not suitable for use in sanitation or healthcare products due to precipitation of silver.

Also, in the reaction as described in (2) above, the interaction of silver oxide with hydrazine hydrate in water is limited by the low solubility of the silver oxide in water. See J. Voigt et al. (Z. Anorg. Allgem. Chem. 164, 409-419, CA21:3512). Therefore, in order to obtain a soluble silver oxide solution, the silver content of the silver oxide solution must be no more than 0.001% by weight. Using such a diluted silver oxide solution as starting material, the resulting silver content in the colloidal silver solution is too low to be effective for sanitation or healthcare use.

The present invention provides a method for making a colloidal nanosilver solution which is distinctively different from the prior art methods. Based on this method, a colloidal nanosilver solution which contains high silver concentration (i.e., containing 0.001% to 0.4% by weight of silver), high stability in the colloidal state (i.e., stable at room temperature for no less than 110 days), and no toxic substances, is formed.

The method for preparing the colloidal nanosilver solution of the present invention contains the following reactive steps:

(1) Dissolution of Silver Oxide in Ammonia Water (N₃.H₂O).

Silver oxide (Ag₂O) is dissolved in concentrated ammonia water (NH₃.H₂O) to obtain a silver ammino oxide [Ag(NH₃)⁺]₂O solution where the silver ion is in the form of silver ammino ion [Ag(NH₃)⁺] in as follows:

Ag₂O+2NH₃.H₂O→[Ag(NH₃)⁺]₂O+2H₂O

The concentrated ammonia water is preferred to be about 28%. The preferred ratio of silver oxide and ammonium water is at about 1:7 to about 1:10, w/v. This procedure has the advantage of increasing the solubility and concentration of silver in the solution.

(2) Dissolution of Gelling Agent in Water to Form a Gelling Medium.

A gelling medium is provided by dissolving a gelling agent in water. This gelling medium serves as a protective gel/colloid mechanism for keeping the nanosilver particles suspended in the colloidal nanosilver solution and preventing the nanosilver particles from aggregating with each other to form bigger pellets and precipitate. Preferably, the concentration of the gelling agent is between 0.2% to 5% by weight.

The gelling agent can be a synthetic or natural polymer or a combination thereof, which can be readily dissolved in water. Examples of the gelling agent include, but are not limited to, starch or starch derivatives, cellulose or cellulose derivatives, polymer or copolymer of acrylate or acrylate derivatives, polyvinyl pyrrolidone (PVP), alginic acid, and xanthogenated gel. The starch derivatives include, but are not limited to, sodium carboxymethyl starch, hydroxyethyl starch, and pre-gelatinized starch. The cellulose derivatives include, but are not limited to, methyl cellulose, hydroxypropyl cellulose, hydroxypropyl methyl cellulose, and hydroxyethyl cellulose. The polymer or copolymer of acrylate or acrylate derivative is preferred to be Carbomer.

Carbomer is a polymer of acrylic acid (or a carboxy vinyl polymer). It is currently sold under the tradename of Carbopol by B. F. Goodrich Company. The preferred carboxy vinyl polymer is a high molecular weight (preferably MW above 1,000,000; and most favorably MW above 3,000,000) polymer, such as Carbopol 934P which has a molecular weight of about 3,000,000.

Carbopol is the trademark of B. F. Goodrich Company's carboxy vinyl polymers, which generally are high molecular weight (“MW”) polymers (MW above 1,000,000). Specifically, Carbopol 934P has a MW of about 3,000,000 and Carbopol 940 is about 4,000,000. The preferred Carbopol is Carbopol 934P.

(3) Mixing Silver Ammino Oxide Solution with the Gelling Medium.

The silver ammino oxide [Ag(NH₃)⁺]₂O solution is thoroughly mixed with the gelling medium to form a uniformly dispersed silver ammino oxide-gelling solution to be used for the next reaction. The silver ammino oxide-gelling medium is preferred to be controlled at about 0° to 45° C.

(4) Reaction of Silver Ammino Ion with Hydrazine Hydrate.

The silver ammino ion is further interacted with hydrazine hydrate in the presence of oxygen gas to form metallic silver, nitrogen gas, and water as follows:

[Ag(NH₃)⁺]₂O+NH₂NH₂.H₂O+2O₂→2Ag_{(metallic)}+2 N₂+6H₂O

The preferred temperature for the above reaction is at about 0° C.-45° C. The reaction is preferred to be conducted in about 0.5 to 2 hours. The silver ammino oxide and hydrazine hydrate are preferred to be at a ratio of 1:0.087 to 1:0.26 by weight. The nanosilver particles prepared by the reactive steps (1)-(4) have diameter of 1 nm to 100 nm.

Because hydrazine hydrate is toxic, after the completion of step (4), the colloidal nanosilver solution is preferred to be kept in the presence of air for additional 0.5 to 5 hours so that the residue of hydrazine hydrate in the final colloidal nanosilver solution can be decomposed into nitrogen and water by the following oxidative reaction:

NH₂NH₂.H₂O+O₂→N₂+3H₂O

The resulting nitrogen gas and water are non-toxic so that no removal of the side products is necessary.

Moreover, the present invention provides a method for making the colloidal nanosilver solution of high silver concentration, high stability in the gel state, and no toxic ingredients. The above mentioned problems associated with the reaction are solved in the present invention: the solubility of silver oxide and final concentration of silver in the colloidal solution are improved, the colloidal nanosilver is stabilized as the gel state in the solution, and the toxic reactant, hydrous ammonia, is carefully removed from the colloidal nanosilver solution by further decomposition reaction with oxygen in the air. The colloidal nanosilver solution of the present invention is suitable for healthcare purposes and serves as an effective antimicrobial agent.

The following examples are illustrative, but not limiting the scope of the present invention. Reasonable variations, such as those occur to reasonable artisan, can be made herein without departing from the scope of the present invention.

### EXAMPLE 1

### Preparation of the Colloidal Nanosilver Solution of the Present Invention

The colloidal solution containing nanosilver particles of the present invention was prepared according to the following steps:

1. 60 g of sodium carboxymethyl starch 60 g was dissolved in 1600 ml of distilled water. The dissolved sodium carboxymethyl starch solution was added to a 5000 ml flask. The flask was heated to and maintained at 70° C. until the solution became gelatinized. The gelatinized solution was cooled down to room temperature.

2. 3 g of silver oxide was added to and mixed with 22 ml of 28% ammonia water to form a silver ammino oxide (i.e., [Ag(NH₃)⁺]₂O)-solution.

3. The silver ammino oxide solution was then mixed thoroughly with the gelatinized solution of (2) to form a silver ammino oxide-gelling medium.

4. 0.5 g of 80% hydrazine hydrate was mixed with and dissolved in 200 ml of distilled water to form a hydrazine hydrate solution.

5. The hydrazine hydrate solution was added to the flask containing silver ammino oxide-gelling medium. An additional 115 ml of distilled water was then added to and mixed with the rest of the solution. The solution was then reacted at room temperature for 1.5 hours under seal. The flask was then opened to allow the reactants to be in touch with air for additional 3.0 hours.

### EXAMPLE 2

### Preparation of the Colloidal Nanosilver Solution of the Present Invention

The colloidal solution containing nanosilver particles of the present invention was prepared according to the following steps.

1. 1600 ml of distilled water was added to a 5000 ml flask and heated to and maintained at 70° C.

2. 50 g of methyl cellulose was gradually added to the flask containing the heated distilled water. After thorough mixing of the methyl cellulose with the distilled water, the temperature of the solution was gradually reduced to around 30° C. until a gelatinized solution was formed.

3. 3 g of silver oxide was added to and mixed with 22 ml of 28% ammonia water to form a silver ammino oxide solution.

4. The silver ammino oxide solution was then added to and mixed with the gelatinized solution to form a silver ammino oxide-gelling medium.

5. 0.6 g of 80% hydrazine hydrate was dissolved in 200 ml of distilled water to form a hydrazine hydrate solution.

6. The hydrazine hydrate solution was then added to the flask containing silver ammino oxide-gelling medium. An additional 125 ml of distilled water was then added to and mixed with the rest of the solution. The solution was then reacted at room temperature for 1 hour under seal. The flask was then opened to allow the reactants to be in touch with air for additional 4.0 hours.

### EXAMPLE 3

### Preparation of the Colloidal Nanosilver Solution of the Present Invention

The colloidal solution containing nanosilver particles of the present invention was prepared according to the following steps.

1. 1600 ml of distilled water was added to a 5000 ml flask and heated to and maintained at 70° C.

2. 4.5 g of carboxypropyl methyl cellulose was gradually added to the flask containing the heated distilled water. After thorough mixing of the carboxypropyl methyl cellulose with the distilled water, the temperature of the solution was gradually reduced to around 30° C. until a gelatinized solution was formed.

3. 4.5 g of silver oxide was added to and mixed with 33 ml of 28% ammonia water to form a silver ammino oxide solution.

4. The silver ammino oxide solution was then added to and mixed with the gelatinized solution to form a silver ammino oxide-gelling medium.

5. 1 g of 80% hydrazine hydrate was dissolved in 260 ml distilled water to form a hydrazine hydrate solution.

6. The hydrazine hydrate solution was added to the silver ammino oxide-gelling medium and 76 ml of distilled water was further added to and mixed with the rest of the solution. The flask was then sealed and kept at room temperature for about 1 hour.

7. The flask was then unsealed to allow the solution to be in touch with air for 4 hours to obtain the colloidal nanosilver solution of the present invention.

### EXAMPLE 4

### Preparation of the Colloidal Nanosilver Solution of the Present Invention

The colloidal solution containing nanosilver particles of the present invention was prepared according to the following steps.

1. 1600 ml of distilled water was added to a 5000 ml flask.

2. 1 g of polyvinylpyrrolidone (PVP) was gradually added into the flask at room temperature and dissolved therein to form a gelatinized solution.

3. 6 g of silver oxide was dissolved in 44 ml of 28% ammonia water to form a silver ammino oxide solution.

4. The silver ammino oxide solution of step (3) was add to and thoroughly mixed with the gelatinized solution of (2) to form a silver ammino oxide-gelling medium.

5. 1.5 g of 80% hydrazine hydrate was dissolved in 270 ml of distilled water to form a hydrazine hydrate solution.

6. The hydrazine hydrate solution was then mixed with the silver ammino oxide-gelling medium of (4) in the flask with 73 ml of additional distilled water added to and mixed into the rest of the solution. The flask was sealed and kept at 30° C. for 1.5 hours.

7. The flask was unsealed to allow the solution to be in touch with air for 5 hours to obtain the colloidal nanosilver solution of the present invention.

### EXAMPLE 5

### Examination of the Dimension and Stability of the Colloidal Nanosilver Solution

I. Purpose:

The colloidal solution containing nanosilver particles of the present invention was examined for the dimension of the nanosilver particles and stability of the colloidal nanosilver solution over time (days) in terms of suspension by electron microscopy.

II. Method:

In accordance with the standard procedures for JY/T011-1996 transmission electron microscope, JEM-100CXII transmission electron microscope was used under the testing conditions of accelerating voltage at 80 KV and resolution at 0.34 nm. The colloidal nanosilver solutions produced by Examples 1-4 of the present invention were observed for the size and distribution of the nanosilver particles therein. Aliquots of the samples from Examples 1-4 were taken out from the solutions either being freshly made or after being stored at room temperature for 110 days.

III. Results:

For the freshly made colloidal nanosilver samples, the diameters of all the silver particles contained therein were below 35 nm, among which, most particles (37%) had a diameter of 15 nm.

For the colloidal solution stored after 110 days, the diameters of all the silver particles contained therein were kept below 35 nm, among which, most particles (38%) had a diameter of 15 nm.

IV. Conclusion:

The colloidal solution of the present invention containing nanosilver particles which had a size range of 1 nm to 100 nm and was very stable after storage of 110 days at room temperature. There was no visible increase in size of the silver particles contained therein and no precipitation of silver particles. The colloidal solution of the present invention was stable for further processing and adopted for use, storage, and transportation.

### EXAMPLE 6

### Antimicrobial Activity of the Colloidal Nanosilver Solution of the Present Invention

I. Purpose:

The colloidal solution of the present invention was tested for the antimicrobial ability.

II. Method:

Microbial strains tested were Escherichia coli, Methicillin resistant Staphylococcus aureus, Chlamydia trachomatis, Providencia stuartii, Vibrio vulnificus, Pneumobacillus, Nitrate-negative bacillus, Staphylococcus aureus, Candida albicans (ATCC 10231), Bacillus cloacae, Bacillus allantoides, Morgan's bacillus (Salmonella morgani), Pseudomonas maltophila, Pseudomonas aeruginosa, Neisseria gonorrhoeae, Bacillus subtilis, Bacillus foecalis alkaligenes, Streptococcus hemolyticus B, Citrobacter, and Salmonella paratyphi C. These strains were either isolated from clinical cases or purchased as standard strains from Chinese Biological Products Testing and Standardizing Institute.

A typical example of the test, as illustrated by Candida albicans (ATCC 10231), was as follows:

Colloidal nanosilver solutions of examples 1-4 (each contains a concentration of 1370 μg/ml of silver) were tested for its antifungal activity against Candida albicans. The colloidal nanosilver solutions were diluted in distilled water to make the final concentrations of 137 μg/ml, 68.5 μg/ml, 45.7 μg/ml, 34.2 μg/ml, and 27.4 μg/ml. In the control group, no colloidal nanosilver solution was added. Candida albicans was added to each tested and control groups, respectively, and the viability of the fungus in each group was examined 2 minutes after incubation with the colloidal nanosilver solutions of examples 1-4.

Typically, due to the resilience of Candida Albicans, a higher concentration of disinfectant is required to kill or suppress the growth of Candida albicans than for killing bacteria such as Staphylococcus aureus and Escherichia coli.

III. Results:

There was an average of 99.99% killing rate (1.78×10⁶ cfu/mu) for all of the colloidal nanosilver solution tested (Examples 1-4) after 2 minutes of incubation. Among the same example, the most diluted sample demonstrated about the same fungicidal activity as the least diluted one.

IV. Conclusion:

The colloidal solution containing nanosilver particles of the present invention was effective as antimicrobial agent even at a diluted concentration of 27.4 μg/ml of silver.

While the invention has been described by way of examples and in terms of the preferred embodiments, it is to be understood that the invention is not limited to the disclosed embodiments. On the contrary, it is intended to cover various modifications as would be apparent to those skilled in the art. Therefore, the scope of the appended claims should be accorded the broadest interpretation so as to encompass all such modifications.

We claim:

1. A colloidal nanosilver solution comprising
a nanosilver particle with diameter ranged between 1 nm and 100 nm; wherein said colloidal nanosilver solution contains 0.001% to 0.4% by weight of silver.

2. The colloidal nanosilver solution according to claim 1, further comprising a gelling agent.

3. The colloidal nanosilver solution according to claim 2, wherein said gelling agent is starch or starch derivative.

4. The colloidal nanosilver solution according to claim 3, wherein said starch derivative is at least one selected from the group consisting of sodium carboxymethyl starch, hydroxyethyl starch, and pregelatinized starch.

5. The colloidal nanosilver solution according to claim 2, wherein said gelling agent which is cellulose or cellulose derivative.

6. The colloidal nanosilver solution according to claim 5, wherein said cellulose derivative is at least one selected from the group consisting of methyl cellulose, hydroxypropyl cellulose, hydroxypropyl methyl cellulose, and hydroxyethyl cellulose.

7. The colloidal nanosilver solution according to claim 2, wherein said gelling agent is polymer or copolymer of acrylate or acrylate derivative.

8. The colloidal nanosilver solution according to claim 7, wherein said polymer or copolymer of acrylate or acrylate derivative is Carbopol 934P.

9. The colloidal nanosilver solution according to claim 2, wherein said gelling agent is polyvinyl pyrrolidone, alginic acid, or xanthogenated gel.

10. The colloidal nanosilver solution according to claim 2, wherein said gelling agent is at a concentration of 0.2 to 5% by weight of the total solution.

11. The colloidal nanosilver solution according to claim 1, wherein said colloidal nanosilver solution inhibits growth of bacteria, fungi, or chlamydia.

12. The colloidal nanosilver solution according to claim 6, wherein said bacteria, fungi or chlamydia are at least one selected from the group consisting of Escherichia coli, Methicillin resistant Staphylococcus aureus, Chlamydia trachomatis, Providencia stuartii, Vibrio vulnificus, Pneumobacillus, Nitrate-negative bacillus, Staphylococcus aureus, Candida albicans, Bacillus cloacae, Bacillus allantoides, Morgan's bacillus (Salmonella morgani), Pseudomonas maltophila, Pseudomonas aeruginosa, Neisseria gonorrhoeae, Bacillus subtilis, Bacillus foecalis alkaligenes, Streptococcus hemolyticus B, Citrobacter, and Salmonella paratyphi C.

13. A method for making the colloidal nanosilver solution according to claim 2 comprising:
dissolving silver oxide (Ag₂O) in ammonia water (NH₃.H₂O) to form a solution containing silver ammino ion [Ag(NH₃)⁺];
dissolving said gelling agent in water to form a gelling medium;
mixing said silver ammino ion-containing solution with said gelling medium to form a colloidal nanosilver ammino ion-containing solution; and
mixing said colloidal nanosilver ammino ion-containing solution with hydrazine hydrate (NH₂NH₂.H₂O) to form the colloidal nanosilver solution.

14. The method according to claim 13, wherein said gelling agent is at least one selected from the group consisting of starch or starch derivative, cellulose or cellulose derivative, polymer or copolymer of acrylate or acrylate derivative, polyvinyl pyrrolidone, alginic acid, and xanthogenated gel.

15. The method according to claim 14, wherein said starch derivative is sodium carboxymethyl starch, hydroxyethyl starch, or pregelatinized starch.

16. The method according to claim 14, wherein said cellulose derivative is methyl cellulose, hydroxypropyl cellulose, hydroxypropyl methyl cellulose, or hydroxyethyl cellulose.

17. The method according to claim 14, wherein said polymer or copolymer of acrylate or acrylate derivative is Carbopol 934P.

18. The method according to claim 13, wherein said colloidal nanosilver ammino ion-containing solution is mixed with hydrazine hydrate (NH₂NH₂.H₂O) at about 0 to 45° C. for about 0.5 to 2 hours.

19. The method according to claim 13, wherein after the formation of said colloidal nanosilver solution, further comprising a step of contacting said colloidal nanosilver solution with air for about 0.5 to 5 hours.

20. The method according to claim 13, wherein said ammonia water is at a concentration of about 28% by weight.

21. The method according to claim 13, wherein said silver oxide and said ammonium water is at a ratio of about 1:7 to about 1:10, w/v.

22. The method according to claim 13, wherein said silver oxide and said hydrazine hydrate is at a ratio of about 1:0.087 to about 1:0.26, w/v.

23. A colloidal nano silver solution which is prepared according to claim 13.

24. The antibacterial or antifungal agent comprising the colloidal nanosilver solution according to claim 1.

25. The antibacterial or antifungal agent according to claim 24, wherein said antibacterial or antifungal agent is used to treat patients with burn and scald-related skin infection, wound-related skin infection, dermal or mucosal bacterial or fungal infection, surgery cut infection, vaginitis, and acne-related infection.

26. A method for disinfection or sanitation comprising applying the colloidal nanosilver solution according to claim 1 to areas in need of disinfection or sanitation.

27. A method for suppressing bacterial or fungal growth in patients comprising applying the colloidal nano silver solution to said patients.
